(19) United States
(12) Patent Application Publication
(10) Pub. No.: US 2002/0044969 A1
(43) Pub. Date: Apr. 18, 2002

(54) Method for increasing the compressibility of poorly binding powder materials

(76) Inventors: Jerome Harden, Philadelphia, PA (US); Duane Glover, Philadelphia, PA (US)

Correspondence Address: Synnestvedt & Lechner LLP, 2600 Aramark Tower, 1101 Market Street, Philadelphia, PA, 19107-2950, US

(21) Appl. No.: 09/862,269

(22) Filed: May 22, 2001

Related U.S. Application Data
(60) Provisional application No. 60/206,142, filed on May 22, 2000.

(51) Int. Cl. A61K 9/20; B29B 9/00

(52) U.S. Cl. 424/470; 264/6

(57) ABSTRACT

A method is described for the direct compression of particles comprising the steps of subjecting a suspension comprising particles of an active material and a compression excipient in a fluid medium to a pressure force to form a suspension of modified particles, removing said modified particles from said fluid medium to form a dried homogenate; and directly compressing said dried homogenate. The method prepares high active-loaded compression forms by direct compressing a mixture of active material with an excipient composition containing a non-ionic hydrophilic polymer, and a polysaccharide. Also described is a direct compression excipient composition comprising polysaccharide and a non-ionic hydrophilic polymer, which excipient may be combined with an aqueous suspension of the difficult-to-compress active, dried and compressed into a form acceptable for purposes including pharmaceutical applications.

### FIELD OF THE INVENTION

The present invention relates to improvements in formulation processing technology, more particularly improvements in the processing of materials for incorporation into compressible compositions to enable the production of a final compressed form such as a tablet, pellet, bead or the like.

### BACKGROUND OF THE INVENTION

The compressed tablet is one of the oldest and most popular unit forms for oral dosage of medicinal substances. As a result of the introduction of new carriers and compression vehicles, tablets are replacing many forms of pills, powders and capsules. Accordingly, tablets presently represent the largest production volume of all pharmaceutical a trials and to supplements.

The reasons for the widespread use of tablets are apparent, since tablets facilitate: (1) administration of medication in an accurate dose; (2) fast and accurate dispensing with less chance of error and contamination: (3) ease of administration: (4) administration in a form in which the time and area of contact between the active ingredient and the taste buds are reduced, thus obviating the physiological problems associated with the oral administration of drugs that possess a bitter taste and, in the case of coated tablets, with drugs that possess a disagreeable odor; (5) release of drugs at specific locations in the gastrointestinal tract to prevent degradation of drugs sensitive to the low pH environment in the stomach, prevent release of drugs that irritate the gastric mucosa in the stomach, and facilitate local action or preferential absorption at specific sites in the tract: (6) enhanced stability by effecting a marked reduction in the surface of the drug exposed to the environment; (7) rapid production; and (8) economy and ease in storage, packaging and shipping.

The preparation of a solid compressed form containing one or more active ingredients (such as drugs or nutrients such as vitamins) requires that the materials to be compressed into the form possess certain physical characteristics that lend themselves to such processing. Among other things, the material to be compressed must be free flowing, must be lubricated, and, importantly, must possess sufficient cohesiveness to insure that the solid dosage form remains intact after compression.

A tablet is formed typically by pressure being applied to the material to be tableted on a tablet press. A tablet press includes a lower punch which fits into a die from the bottom and a upper punch having a corresponding shape and dimension which enters the die cavity from the top after the tableting material fills the die cavity. The tablet is formed by pressure applied on the lower and upper punches. The ability of the material to flow freely into the die is important in order to insure that there is a uniform filling of the die and a continuous movement of the material from the source of the material, e.g. a feeder hopper. The lubricity of the material is crucial in the preparation of the solid dosage forms since the compressed material must be readily ejected from the punch faces.

Since most drugs and nutritional supplements have none of these properties, methods of tablet formulating have been developed to impart these desirable characteristics to the material(s) which is to be compressed into a solid dosage form. Typically, excipients, which impart good flow and compression characteristics to the material as a whole, are added to the active material that is to be compressed. Such properties are typically imparted to these excipients via a preprocessing step such as wet granulation, slugging, spray drying, spheronization, or crystallization. Useful direct compression excipients include processed forms of cellulose, sugars, and dicalcium phosphate dehydrate, among others.

There are three general methods of preparing thermally solid dosage form prior to compression: (1) dry granulation; (2) wet granulation; and (3) direct compression.

Dry granulation procedures may be utilized where one of the constituents, either the drug or the diluent, has sufficient cohesive properties to be tableted. The method includes mixing the ingredients with a lubricant, if required, slugging the ingredients, dry screening, lubricating and finally compressing the ingredients.

The wet granulation includes mixing the powders to be incorporated into the dosage form and thereafter adding solutions of a binding agent to the mixed powders to obtain a granulation. Thereafter, the damp mass is screened, e.g., in a 6- or 8-mesh screen and then dried, e.g., via tray drying or fluid-bed drying. One disadvantage of the wet granulating technique is that it has been known to reduce the compressibility of some pharmaceutical ingredients including microcrystalline cellulose.

Direct compression is a relatively quick process wherein the powdered materials included in the solid dosage form are compressed directly without modifying their physical nature. Usually, the active ingredient, direct compression vehicle and other ancillary substances, such as a glidant to improve the rate of flow of the tablet granulation and lubricant to prevent adhesion of the tablet material to the surface of the dies and punches of the tablet pressure are blended in a twin shell blender or similar low heat apparatus before being compressed into tablets.

Direct compression is usually limited to those situations where the drug or active ingredient has a requisite crystalline structure and the physical characteristics required for formation of an acceptable tablet. However, only a very limited number of substances possess enough cohesive strength and flowability to allow direct compression without previous granulation. A limited number of crystalline materials, such as potassium bromide and potassium chloride, can be compressed without preliminary treatment. Also, drugs such as aspirin and phenolphthalein can be directly compressed after blending with suitable tableting excipients.

It has been estimated that about 20 percent of the materials used for tableting in the pharmaceutical field may be compressed directly. In order to use this method to a greater extent, many more materials are modified either by treating the material in some special way during early stages of preparation, or by adding a direct compression vehicle, i.e., a dry binder or excipient material which will mix with the active ingredient to provide a flowable powder and form an easily compressible carrier.

There are currently several available binders or excipients that can be used as direct compression vehicles. They include spray-dried lactose; anhydrous lactose: microcrystalline cellulose; dicalcium phosphate dehydrate, unmilled; spray-congealed mannitol; ungelatinzed starch (e.g., cornstarch), and partially or fully pregelatinized starch.

Microcrystalline cellulose, processed cellulose, has been utilized extensively in the pharmaceutical industry as a direct compression vehicle for solid dosage forms. Microcrystalline cellulose is commercially available under the tradename Emcocel® from Edward Mendell Co., Inc. and as Avicel® from FMC Corp. Compared to other directly compressible excipients, microcrystalline cellulose is generally considered to exhibit superior compressibility and disintegration properties as long as it is not wet granulated prior to compression.

Many types of partially or fully pregelatinized starches are commercially available for use in direct compression tablet formulations. Pregelatinized cornstarch provides tablets with hardness properties in the range of 1 to 4 Kp. Present demands, however, require hardness levels in the range of 10-14 Kp and higher, an expectation which many starches modified by prior art methods simply can not meet. While the use of starch in tableting formulations is still common practice, problems of uniformity between modified batches and a demand for tablets of greater hardness resulted in its departure from the status of a preferred pharmaceutical excipient.

The prior art also discloses inherently cost ineffective chemically modified starches requiring the additional expense of crosslinking chemicals or functional reagents to produce the desired physical characteristics in the substrate. Disposal problems associated with unwanted reaction by-products further adds to cost and environmental concerns. Also, chemical modification methods yield product in batch quantities, rather than on a continuous or semi-continuous basis and, therefore, are less time efficient. Production rates are further diminished when more than one chemical modification must be made to the starch substrate to yield a product with all of the desired characteristics. Moreover, the starch end product itself often suffers from other limitations similar to the deficient tablet hardness profiles, discussed above. Inferior viscosity, shear resistance and thermal profiles of the starch end product, for example, may frustrate the performance of products incorporating starch modified by prior art means.

Since each excipient added to a formulation necessarily increases the tablet size of the final product, compression techniques are often limited to formulations containing a rather low load of active ingredient per compressed tablet. Solid dosage forms containing the active ingredient to be administered in a relatively high load or dose (e.g., the drug itself comprises a substantial portion of the total compressed tablet weight), could only be directly compressed if the drug itself had sufficient physical characteristics ( e.g., cohesiveness) for the ingredients to be directly compressed.

For example, acetaminophen, a widely used analgesic, is considered to be a high load active ingredient. Most commercial compressed tablet formulations include anywhere from 70 to 85% be weight acetaminophen per finished tablet. This high load of active ingredient combined with its rather poor physical characteristics for direct compression has not allowed pharmaceutical manufacturers to use direct compression techniques to prepare the final tablets.

Thus, another limitation of direct compression as a method of tablet manufacturing is the potential size of the compressed tablet. If the amount of active ingredient is high, a pharmaceutical formulator may choose to wet granulate the active with other excipients to attain an acceptably sized tablet with the desired amount of active ingredient, such as acetaminophen. Usually the amount of filler/binder or excipients needed in wet granulation is less than that required for direct compression since the process of wet granulation contributes to some extent toward the desired physical properties of a tablet. Even so, other reasons, such as for example the loss of compressibility associated with the wet granulation of MCC, prevent wet granulation from always being the solution to increase active loading and reduce the size of compressed tablet.

### REPORTED DEVELOPMENTS

Exemplary U.S. patents relating to directly compressible tablets include U.S. Pat. No. 3,584,114 to Cavalli, et. at., U.S. Pat. No. 3,725,556 to Hanssen, et al., U.S. Pat. No. 3,873,694 to Kanig, U.S. Pat. No. 4,072,535 to Short, and U.S. Pat. No. 4,439,453 to Vogel.

U.S. Pat. No. 5,455,342 discloses starch and other polymers treated with high pressure using a piston apparatus resulting in a starch end-product that manifests several changes in physical properties, including: an altered thermal profile (the onset of melting and the actual melting point is raised, the heat energy required to effect melting is also altered); altered disintegration and solubility properties (the solubility rate in water and other solutions in an ambient or heated environment is slowed by as much as 300%); and altered viscosity profile (pressure treated starch exhibits a higher viscosity for a longer period of time); an altered tableting profile (the treatment of waxy maize pre-gelatinized starches results in a starch which forms harder tablets at lower than conventional compression forces); and an altered turbidity profile (the clarity of solutions made with pressure treated starch is improved).

U.S. Pat. No. 5,455,342 discloses also that pressure treated starch samples are useful as excipients in tableting processes in view of the need for a more readily dissolvable excipient than microcrystalline cellulose. The '342 patent discloses the direct compression preparation of pharmaceutical tablet compositions including acetaminophen and vitamin C where microcrystalline cellulose (MCC) and untreated starch are replaced with equal amounts of the pressure treated starch. Resulting tablets exhibit acceptable friability, high hardness, and slower dissolution rates than the MCC containing compositions.

U.S. Pat. No. 4,950,484 discloses antibiotic compositions that are wet granulated and compressed and are characterized as including a high percentage of active antibiotic which can be 20-70 wt %, but is preferably 50-65 wt %. These compositions contain a substantial amount of MCC or microfine cellulose in combination with a disintegrant. The '484 patent disclose also that 20-50 wt %, preferably 35 -45 wt % based on the weight of antibiotic of microcrystalline and/or microfine cellulose is used in the granulate, while further amounts, 4-20 wt%, preferably 8-15 wt % based on the weight of the antibiotic, of microcrystalline and/or microfine cellulose are then added to the granulate. These compositions also include 2-20 wt %, preferable 7-10 wt %, based on the weight of the antibiotic of a low-substituted hydroxypropylcellulose as a disintegrant.

U.S. Pat. No. 5,137,730 discloses an improved tablet composition for drugs or active ingredients prone to poor tableting properties. Although the '730 patent discloses that the premixture used in wet granulation consists essentially of between about 85 and 99.9 percent by weight of the active ingredient and between about 0.1 and 15 percent by weight of citric acid, and one or more other formulation ingredient added to the premixture, the final compressed includes substantially more excipients in the product.

Thus, there still remains a need in the industry for techniques and formulation excipients which would allow artisans to prepare direct compression dosage forms containing relatively high amounts of weight of active ingredient(s) such as for example acetaminophen and vitamin formulations.

Vericon Inc. of Lionville, Pa., formerly known as Delta Food Group, Inc. of Aston, Pa., sells the commercially available product, Del Tab™ excipient which is manufactured in accordance with the teachings of U.S. Pat. No. 5,455,342. This product, previously made available under the mark, Delta Starch®, possesses superior properties to microcrystalline cellulose and standard starches used in pharmaceutical and nutritional supplement formulations. The Delta Starch product literature describes that Delta Starch (otherwise identified ad DS-901), versions A and C, produce very hard tablets at low compression pressures. For example, a 200 mg tablet made of pure DS-901,pressed at just 1 ton, will possess an average hardness of 20 Kp, and that ideal applications include chewable tablets where mouth feel, tablet strength, yet a good friability are required. DS-901-C is described as ideal for direct compression where its performance is comparable to microcrystalline cellulose in most tableting operations. As a wet binder DS-901-C is described as superior to all other conventional pharmaceutical grade starch binders and is also superior to Microcrystalline Cellulose (MCC), requiring less time and less moisture to provide it's binding function, while also enabling a significant tablet hardness, with friability comparable to Starch 1500 or MCC variations. DS-901-C is described as functioning as an anti-caking agent when used in loading levels from 10 to 35% but also provides a higher tablet hardness than other starches. The product literature describes the following examples of tablets produced with DS 901-C: Formulation VIT-101 used DS-901-C as the diluent and wet binder with Vitamin C. Compared to Starch 1500, DS-901-C used far less moisture and reduced the wet granulation time by half, yet retained tablet hardness, friability and appearance. Formulation VIT-102 used DS-901-C as a direct compression diluent for an extremely hygroscopic compound, choline chloride crystal. DS-901-C provides longer shelf life and greater stability over MCC in this application.

Delta Starch product literature also describes DS901-B as used as a wet binder for pharmaceutical tableting, and is particularly used as a flow control binder for such products as acetaminophen and ascorbic acid formulations which may use from 10 to 35% starch as a binder in fluid bed granulation processing. DS901-B provides excellent binding, while using as much as 30% less water during fluidizing processing, and enabling as much as 50% shorter overall processing times in a wet granulation of fluid bed processing procedure. Additionally the DS-901-B ingredient after wet granulation provides improved compressive strength, higher tablet hardness, and a reduction in the need for critical diluents such as microcrystalline cellulose or lactose, while providing strong tablets with reduced capping and friability. Nonetheless, the product literature does not recognize or suggest that the Delta Starch product could eliminate substantially all of the diluent, binders and hardening agents required in prior art tableting formulations and significantly reduce tablet size while retaining desired tablet properties.

The present invention is based on the surprising discovery that the processing of difficult-to-compress active ingredients in admixture with small amounts of tableting excipients, previously included in formulations at higher than 25% by weight, may be used in preparing active materials for incorporation into direct compression formulations. The present discovery enables the direct compression manufacture of reduced size high active loading tablets which otherwise would be difficult and/or expensive to prepare with the standard methods.

### SUMMARY OF THE INVENTION

The present invention relates to a process for increasing the percentage of active ingredient relative to non-active excipient in a compressible formulation by taking the following steps:

(1) subjecting a suspension comprising particles of an active material and a compression excipient in a fluid medium to a pressure force to form a suspension of modified particles;

(2) removing said modified particles from said fluid medium to form a dried homogenate; and

(3) directly compressing said dried homogenate.

The process may be used to form the reduced volume mixture into an object such as a tablet, a pellet, sphere, disk or any shape susceptible to compression formation, such as in tablet stamping or extrusion pelleting. Exemplary excipients that may be replaced or substantially reduced in amount by using the process of the present invention include silica, cellulose, microcrystalline cellulose, calcium carbonate, mono-, di- or tri-calcium phosphate, spray-dried lactose, anhydrous lactose, dicalcium phosphate dihydrate, unmilled, spray-congealed mannitol, ungelatinized starch (e.g., corn starch), and partially or fully pregelatinized starch. The resulting product is particularly useful as a nutritional supplement or a pharmaceutical composition suitable for animal or human ingestion.

Another aspect of the present invention relates to a process for reducing the size of a solid compressed tablet which size would be about 30% greater if such tablet containing a difficult-to-compress active ingredient were prepared by the prior art wet granulation method, comprising the steps of:

(1) preparing a suspension of said difficult-to-compress active ingredient in a first amount and a polysaccharide-containing excipient in a second amount in an aqueous medium;

(2) wherein said second amount is at least about 30% less than a third amount that is required by a wet granulation process to form a compressible form with said active ingredient; and

(3) subjecting said suspension to sufficient pressure, shear and/or cavitation forces such that the resulting suspendant particles, after drying, are capable of direct compression into USP acceptable tablets;

(4) recovering said particles from aqueous medium;

(5) drying and compressing said recovered particles to form a USP acceptable tablet.

A particularly preferred embodiment of the present invention is the preparation of high active-loaded compression form by combining a non-ionic hydrophilic copolymer, a polysaccharide and the active ingredient in amounts such that the active is present in amounts greater than about 92% in the final form, which exhibits acceptable hardness, friability and disintegration properties.

Another aspect of this invention is the preparation of a compression excipient comprising polysaccharide and a non-ionic hydrophilic polymer, which excipient may be combined with an aqueous suspension of the difficult-to-compress active, dried and compressed into a form acceptable for purposes including pharmaceutical applications.

Advantages of the present invention include reduced cost of production, improvements in direct compression processing, reduction in patient dosing, reduced tablet size to facilitate dosage acceptability and reduction in choking hazards.

Reduced size tablets produced by the present invention may be manufactured for human or animal consumption and exhibit a hardness and friability within acceptable consumption ranges.

### DETAILED DESCRIPTION

The following terms shall have the meanings as described below:

“Binders” are agents that impart cohesive qualities to the powdered material(s). Commonly used binders include acacia, alginic acid, alkali metal alginate, carbomer, carboxymethylcellulose sodium, dextrin, dicalcium phosphate, dihydrate, ethyl cellulose, gelatin, glucose, guar gum, hydroxyethyl-, hydroxypropyl- and hydroxypropyl methyl-cellulose, hydrogenated vegetable oil, spray-dried lactose; anhydrous lactose, magnesium aluminum silicate, maltodestrin, methylcellulose, microcrystalline cellulose, unmilled; spray-congealed mannitol; povidine, starch (e.g., corn starch), partially or fully pregelatinized starch, and zein.

“Compressible” means a mixture of particles that is capable of forming a tablet after compression and does not remain in a powdered or substantially powdered form or mixture of agglomerated fragments.

“Dextrin” means a mixture of polysaccharides having an empirical formula, (C₆H₁₀O₅)ₙ.xH₂O derived from the heat degradation or particle hydrolysis of starch. To be categorized as a Dextrin, the US Pharmacopoeia publishes limit specifications including a a bulk density less that 0.8 g/cm3, a tap density of less than 0.91 g/cm3 and a particle size distribution where about 100% of the particles are less than 60 microns. Dextrins may be produced in a dry reaction, pyrolysis, in the presence of acid or result from the degradation of any aqueous slurry or solution of the starch subjected to high pressure treatment in accordance with the process described in U.S. Pat. No. 5,455,342.

“Diluent” is frequently added in order to increase the bulk weight of the material to be tableted in order to make the tablet a practical size for compression. This is often necessary where the dose of the drug is relatively small.

“Disintegrant” is often included in order to ensure that the ultimately prepared compressed solid dosage form has an acceptable disintegration rate in an environment of use (such as the gastrointestinal tract). Typical disintegrants include starch derivatives and salts of carboxymethylcellulose.

“Friability” is related to the integrity of the tablet and is represented as the percentage of tablet weight loss occurring after a certain number of revolutions in a Vanderkamp Friabilator. The highest integrity tablet has the lowest percentage friability.

“Hardening agent” means an excipient that is incorporated into a compressed tablet composition to impart increased hardness thereto. Exemplary hardening agents include calcium carbonate, di- and tri-calcium phosphate, calcium sulfate, microcrystalline cellulose, powdered cellulose, dextrates, dextrin, sugars such as dextrose, fructose, lactose, mannitol, sorbitol, sucrose, glyceryl, palmitostearate, kaolin, magnesium carbonate, magnesium oxide, maltodextrin, potassium chloride, sodium chloride, starch, pregelatinized starch, talc and hydrogenated vegetable oil.

“Hardness” of the tablet is the force in kp (kilopound) or SCU (1 SCu=1.4 kp) required to break a tablet. The strongest tablet has the highest kp value.

“Lubricants” are typically added to prevent the material(s) being tableted from sticking to the tablet press punches. Commonly used lubricants include magnesium stearate, stearic acid and calcium stearate.

“Maltodextrin” is a mixture of polysaccharides resulting from acid or enzyme hydrolysis of common corn starch or waxy maize starch. The mixture consists principally of D-glucose units linked primarily by alpha 1-4 bonds and is characterized by a relatively uniform distribution of polymeric polysaccharide species.

“Polysaccharide material” as used herein means an organic polymeric material considered as derived from aldose or ketoses by condensation polymerization, composed of repeating monosaccharide units, and are branched or straight-chained, looped or coiled. An exemplary polysaccharide derived from hexoses has the general formula (C6H10O5)n. Polysaccharide materials as meant herein include those materials that are, or may in the future be, approved for human consumption as published by the US Food and Drug Administration. Preferred polymeric materials include the dextrins and maltodectrins, as well as the polysaccharide materials identified in U.S. Pat. No. 5,455,342, hereby incorporated by reference.

“Pregelatinized starch” is defined by the National Formulary XVI as “starch that has been chemically and/or mechanically processed to rupture all or part of the granules in the presence of water and subsequently dried. Some types of pregelatinized starch may be modified to render them compressible and flowable in character.”

“Starch” means a natural polymer (C₆H₁₀O₅)ₙ derived from plant materials, and is commonly found in the form of tiny microscopic granules(5-25 microns in diameter) comprised of stratified layers of starch molecules formed around a hilum nucleus. The starch granule may b round, oval or a angular shape, and consists of a radially oriented crystalline aggregate of two anhydrous D-glucose polymers: amylose and amylopectin. The former is a straight chain polymer of several hundred glucose units linked by alpha-1-2-glycosidic linkages. Amylocpectin is a branched polymer of several thousand glucose units with alpha-1-6-glycosidic linkages at the branched points and alpha-1-4 linkages in the linear regions. Individual branches may have between 20-30 glucose residues. The National Formulary XVI defines Starch as “consist[ing] of the granules separated from the mature grain of corn{Zea mays Linne (Fam.Gramineae)} or of wheat {Triticum asetivum Linne (Fam.Graminaea)}, or from tubers of the potato {Solanum tuberosum Linne (Fam.Solanaceae)}.”

“Tablet” as used herein is intended to encompass compressed nutritive and pharmaceutical dosage formulations of all shapes and sized, whether coated or uncoated. Substances that may be used for coating include hydroxypropylmethylcellulose, hydroxypropylcellulose, titanium oxide, talc, sweeteners and colorants.

“Abrupt pressure treated” materials useful in the practice of the present invention are preferably the pressure treated polysaccharide materials described generally in U.S. Pat. No. 5,455,342, hereby incorporated by reference.

A preferred aspect of the present invention is the preparation of compression formulations including high percentages of active ingredients that exhibit poor binding ability. Such ingredients are typically characterized as crystalline powders, and include, among others, acetaminophen, ascorbic acid, pseudoephedrine, and ibuprofen.

A preferred method according to the present invention involves the preparation of a suspension of active ingredient particles in a fluid medium, most preferably an aqueous medium, with a polysaccharide excipient in an amount equal to about 3 to about 18% by weight of the final dried formulation. A more preferred embodiment of this process utilizes a water insoluble active ingredient particle and a water soluble polysaccharide excipient.

A most preferred method comprises subjecting an aqueous suspension containing a difficult-to-compress water insoluble active material in an amount of about 92 to about 97 wt percent of the total dried materials, a polysaccharide material in an amount from about 3 to about 6 wt percent of the total dried materials, and a non-ionic hydrophilic polymer material from about 0.5 to about 2 wt percent of the total dried materials, to shear, pressure and/or cavitation forces sufficient to intimately admix said materials, removing the water from said composition resulting in a dried admixture, and directly compressing the dried admixture into a compressed form acceptable for purposes including pharmaceutical applications.

A most preferred embodiment of the present method subjects the suspension to a pressure-pulse means to impart shear, abrupt pressure and/or cavitation forces sufficient to intimately admix said active material and said excipient. The application of the pressure-pulse may be accomplished by means of a hydraulic pump as described in U.S. Pat. No. 5,271,881. A preferred apparatus includes a pressure reduction chamber through which the suspension is forced and in which shear and/or cavitation forces impact the suspension.

Applicants believe that the present process micronizes, and modifies the surface characteristics of, the active ingredient particle. Applicants theorize that the high pressure pulse process generates a shock wave through the liquid media containing dissolved polymers and suspended active solids, which shock wave causes the dissolved polymer to go through a phase change to a semi-suspended solid. This phase change will deposit polymer onto the suspended active solid and creates, when dried, a material that exhibits improved compressible properties. In some cases, this phase change may cause the materials to undergo physical changes, such as, changes in viscosity, tensile strength, bulk density, among others. Not all materials will go through this phase change at the same pressure. Different polymers may be effected at different pressures, Applicants theorize that by applying small amounts of the excipient onto the active surfaces, adherent contact points, as well as agglomerates are formed, both of which enhance the binding properties of the active. Applicants do not believe that encapsulation of the active ingredients is occurring due to the relatively small amounts of excipient composition utilized and the relatively large surface area of the active particles.

In this pressure process, described in U.S. Pat. No. 5,455,342, a liquid substrate composition comprising the material and characterized as either a solution, slurry, dispersion, emulsion, mixture, suspension, or other substance exhibiting fluid dynamics, is treated by an apparatus wherein a shock wave transmitting extreme heat and force is applied to the substrate thereby resulting in its modification. The preferred apparatus utilizes a piston that impacts the liquid substrate with forces theoretically ranging from about 13,000 psi to 300,000 psi thereby modifying the physical properties of the material. The preferred actual pressure range is from about 3,000 psi to 8,000 psi, with the most preferred pressure range of about 4,000 to about 8,000 psi.

The pressure-pulse may be applied to the suspension once, twice or as many times as needed to achieve the intimate admixture. A most preferred process subjects the suspension to at least two pressure pulse treatments.

After pressure treatment, the fluid medium is removed from the treated suspension. The fluid may be removed by any means including the application of heat, or vacuum, or by using spray drying or freeze drying techniques. The preferred drying means is spray drying. When using the to spray drying technique, care must be taken to chose a spray drying nozzle that will be large enough to permit the formation of relatively large particle sizes or agglomerates. Such larger particle sizes will facilitate the acceptable flow rates required to feed the compression chambers used in rotary tablet presses.

The resulting dried homogenate may be milled prior to compression if dried in a fluid bed, drying drum, vacuum or in an oven. The dried homogenate is then combined with lubricants and introduced into a tablet press.

By using the present method, the compressed tablet may comprise from about 82 to about 99 percent active ingredient and from 1 to about 15 percent excipient composition.

A special embodiment of the present process uses an excipient composition containing from 100 to about 85% polysaccharide and 0 to about 15% of a non-ionic hydrophilic polymer, and most preferably about 90 to about 38 weight percent of polysaccharide to about 10) to about 40 weight percent of a non-ionic hydrophilic polymer. These materials must be capable of replacing the bulk of standard tableting excipients in a direct compression tableting mixture and of imparting to a compressed tablet, incorporating said preferred polysaccharide in an amount from 1 to about 10 percent by weight, a tablet hardness from 6 kp to about 40 kp, and acceptable friability and an aqueous disintegration time of greater than about 30 seconds and less than 30 minutes in accordance with USP 701. The preferred method results in the formation of USP acceptable tablets with a hardness from about 7 to about 30 kp.

Many hydrophilic polymers many be used in the present process, such as those described in U.S. Pat. No. 5,271,881, hereby incorporated by reference. Preferred polymers useful in achieving the high active loading tablets containing difficult-to-compress active ingredients are non-ionic hydrophilic polymers. The most preferred polymers in this class comprise a hydrophobic backbone and pendent groups imparting hydrophilic character to the polymer. Such pendent groups may be cyclic or acyclic and include nonionic polar moieties such as carbonyl and amide functional groups. Preferred pendant groups include cyclic carbonyl groups such as cyclic ketones, lactones and lactams. The most preferred groups include lactams containing four to ten member rings. Exemplary preferred pendant lactam groups include pyrrolidone, valerolactam, caprolactam, omega-lactam, and betalactam. The most preferred polymers comprise the hydrophobic backbone bonded to the nitrogen of the cyclic lactam. The special embodiment of polymer useful in the present invention is polyvinylpolypyrrolidone (N-vinylbutyrolactam polymer; poly[1-2(2-oxo-1-pyrrolidynyl)ethylene]),

The preferred polysaccharide materials useful in the practice of the present invention include the modified starches, dextrins and maltodextrins. The most preferred polysaccharide materials useful in the practice of the present invention include modified starches such as Capsul® brand modified starch, derived from waxy maize and sold by National Starch and Chemical Company., and abrupt-pressure treated starches, some of which may be characterized as dextrins. A special embodiment of this dextrin material comprises a starch treated with the apparatus disclosed in the '342 patent, available commercially as DEL TAB™ excipient (Drug Master File Number: DMF 10572, November 1993). Other excipients include Delta Cellulose™ excipient, and the various pressure treated polysaccharide materials disclosed in US Patent 5,455,342.

It is understood that certain active ingredients are easily compressible and require little if any binder or hardening agent. Examples of such ingredients may include acylclovir, aspirin and dextromethorphan.

For those active ingredients that are moderately compressible, examples of which include flurbiprofin, citric acid and chlorpheniramine maleate, the resulting tablet composition may include from about 1 to about 4 percent of excipient and most preferably about 1 to about 3 percent of excipient.

For those active ingredients that are moderately uncompressible, examples of which include vitamins, such as vitamin D, calcium citrate malate, the resulting tablet composition may include from about 2 to about 6 percent of excipient and most preferably about 2 to about 4 percent of excipient.

For those difficult-to-compress ingredients, examples of which include acetaminophen, ascorbic acid, pseudoephedrine, and ibuprofen, the present process offers the unexpected benefit of providing high active loading compositions including such ingredients using direct compression processing. For these difficult to compress ingredients, the resulting tablet composition may include from about 3 percent up to about 18 percent of excipient composition and most preferably from about 5 percent to about 10 percent of excipient, and most preferably from about 5 to about 7 percent of excipient.

The formulations compressed in the practice of this invention and the tablet compositions of the present invention may also contain one or more additional formulation ingredients that may be selected from a wide variety of excipients known in the formulation art, in particular the pharmaceutical art. According to the desired properties of the tablet, any number of ingredients may be selected, alone or in combination, based upon their known uses in preparing tablet compositions. Such ingredients include, but are not limited to disintegrants, lubricants, flavors, flavor enhancers, sweetener, colors and preservatives. Some of these ingredients are included to aid in dissolution, consumption acceptability such as taste, texture and color of the compressed form, while others aid in the processing of the product, by improving powder flowability. It is understood that these further excipients are to be included in small amounts to provide the desired function and not in amounts that may appear to frustrate the purposes of the present invention. Accordingly, such excipients should not be included in amounts of more than about 5% by weight of the final tablet composition. Most preferably, these further excipients are to be included in amounts of about 0.1 to about 2 percent by weight of the final composition.

Processes for tableting are well known to those skilled in the art. Modern compression tableting techniques—irrespective of the type (and ultimate shape of the end product)—utilize a piston like device with three stages in each cycle: (1) filling—adding the powder constituents of the tablet to the compression chamber; (2) compression—forming the tablet; and (3) ejection—removing the tablet. The cycle is then repeated. Representative tablet presses are the Manesty Express 20 rotary press, and Manesty D3A Dry Cota rotary tablet press manufactured by Manesty Machines Ltd., Liverpool, England, and the Key International Rotary tablet Press manufactured by Key International, Englishtown, N.J. It will be appreciated by those skilled in the art that other tableting machines capable of compressing a tablet can also be used.

In order to make tablets, preferably all ingredients—or at least the carrier or hardening agent which typically makes up the bulk of the tablet—must have certain physical characteristics, including the ability to flow freely, and acceptable cohesion (or compressibility). Because many materials have some, or none, of these qualities, techniques must be developed to impart these characteristics to the constituents. In this context, “free flowing” means that the particles to be compressed must enter the compression chamber as discreet particles. While particles which are not “free flowing” can be used in tableting contexts, they can be utilized only if force feeders or other mechanical means are utilized to move the particles, To facilitate the free-flowing nature of the particle mixture, the ingredient mixture may be milled prior to introduction into the tablet press. Sometimes a lubricant is added to the formulation. Preferred lubricants useful in the practice of the present invention include magnesium stearate and stearic acid. Such lubricants are commonly included in the final tableted product in amounts usually less than about 1% by weight based on the total weight of the dried composition.

Two critical criteria in the quality of a tablet are crushing strength (or hardness) and friability. The resistance of the tablet to chipping, abrasion, or breakage under conditions of storage, transportation and handling before usage depends on its hardness. Tablets of insufficient hardness exhibit capping and/or lamination and can easily break apart or disintegrate under normal handling and packaging conditions. Tablets of insufficient hardness cannot be used for lozenges or mints which are designed to be sucked in the mouth, releasing the active ingredient(s) or flavor over time, and may have an undesirable powdery, grainy or coarse mouthfeel.

Hardness is measured by determining lateral breaking strength (expressed in kiloponds or Strong Cobb Units wherein 1 kp=1.4 S.C.U,) exerted on a single tablet at the moment of rupture. Acceptable hardness depends on the desired mouthfeel and the expected end use and packaging conditions of the tablet, but in most contexts, tablet hardness must be greater than about 7 kp to be commercially useful.

Friability is also a standard test known to one skilled in the art. Friability is measured under standardized conditions by weighing out a certain number of tablets (generally 20 or more), placing them in a rotating Plexiglas drum in which they are lifted during replicate revolutions by a radial louver, and then dropped through the diameter of the drum. After replicate revolutions, the tablets are reweighed and the percentage of powder “rubbed off” or broken pieces is calculated. Friability in the range of about 0% to 3% is considered acceptable for most drug and food tablet contexts. Friability which is about 0% to about 1% is preferred, while 0% to 0.6 % is more preferred, while friability less than about 0.3% is most preferred.

Using various compression forces (0.1 to 6.0 tons), tablet hardness limits of 5.0 SCU to 11.0 SCU (7kp to 12 kp), 10.0 SCU to 14.0 SCU (14 kp to 19 kp)and14.0 SCU to 28.0 SCU (19 kp to 39 kp) for various types of formulations, respectively, will provide acceptable results. Typically, formulations of the present invention are compressed using about 0.5 to about 3.5 ton pressure settings. These hardnesses result in acceptable adherence of the press coat to the core tablet for all three products, with no picking, capping or lamination. When the compositions are compressed within those hardness limits, broken or chipped tablets are minimized or eliminated, and weight loss is expected to be less than one percent. Tablet thicknesses are within five percent of the average thicknesses and tablet dissolution results are well within the specification of Q=75% in 45 minutes according to the U.S. Pharmacopoeia National Formulary.

A variety of tablets may be produced by the present invention. Depending on the hardness, and palette acceptable flavoring and lubricant excipients included in the formulation, the tablet may be either chewable or swallowable.

Chewable tablets produced according to the present invention exhibit a hardness within the range of about 5 SCU to about 11 SCU (7 kp to 15 kp). A most preferred range is between about 7 to about 9 SCU (9 kp to 12 kp).

Swallowable tablets may exhibit a hardness within the range of about 5 SCU to about 28 SCU (about 7 kp to about 39 kp), with a preferred hardness between about 7 to about 20 SCU (10 kp to 30 kp). A most preferred range is between about 10 to about 15 SCU (14 kp to 22 kp).

Whether the present process produces a chewable or swallowable tablet, the tablets friability is within the range of about 1% to 0%, preferably 0.6% to 0 %, and most preferably 0.3 to 0%.

Swallowable tablets made according to the present invention may be formulated to disintegrate in more than 20 seconds and in less than 30 minutes in accordance with USP Specification 701. The minimum disintegration time of 20 seconds permits the consumer sufficient time to swallow a whole tablet and avoid in-mouth dissolution. Preferred dissolution times are greater than about 40 seconds, more preferably greater than 60 seconds and most preferably more than about 90 seconds to permit the tablet to travel the esophageal channel intact. If the disintegration time is less than 30 seconds, the surface integrity of the tablet is compromised in the mouth providing the dissolution of the surface layer after a few moments residence time. Such tablets are typically coated thinly to mask the taste of the active ingredient from the consumer. It is preferred that tablets made according to this invention maintain their surface integrity for more than about 15 seconds to avoid the necessity for a taste masking coating. It is believed that such surface integrity properties are achieved with a disintegration time of about 40 seconds or more.

### TESTING APPARATUS AND METHODS

Tablet hardness is determined by using the Schleuniger Hardness Tester made by Dr. K. Schleuniger & Co. Another representative hardness tester is the Model HT-300 manufactured by Key International, Inc., and the Vector Corporation Tablet Hardness Tester, Model #Computest.

Tablet friability is determined by using the Roche friability method (Remington's——Pharmaceutical Sciences, Ed. 1980, page 1558—Mack Publishing Company) and a friability tester, particularly the Distek Friabilator.

Dissolution time of the active ingredient is determined for all the tablets by the method described in the USP XXII, page 683 and by using the equipment described in the USP XXII, pages 1578-9, Apparatus 1. An exemplary commercial system is the Distek Dissolution System 2100B, Model 2100B.

Disintegration time for the tablets, coated tablets and capsules prepared according to the present invention is evaluated according to the method described in the USP XXII, pages 1577-1578 (Procedure 701). An exemplary commercial system is the Distek Disintegration Tester, Model ED-2.

The invention is illustrated in the following examples. The examples do not limit the scope of the invention in any manner. All percentages and ratios are by weight unless otherwise stated.

### EXAMPLE 1

Table 1 presents data on tablets incorporating difficult-to-compress materials prepared using the present invention in comparison with prior art methods. All preparations listed in Table 1 were prepared using the same amounts of lubricant (magnesium stearate) to facilitate the tablet punching operation and of croscarmellose sodium (AcDiSol®, NF) to aid in disintegration. The tablets were pressed using a Key International Rotary Press DC-16 fitted with (four) ½ inch concave beveled edge tooling, a fill gauge setting of 12 mm and a speed set at 18 rpm. The Table identifies the drying method used: either oven dried until the moisture levels were reduced to between about 2 to 8% by weight, or spray dried.

All examples described in Table 1 were prepared without observing the capping or lamination of Flow.. the tablets during friability testing. The Table identifies the active ingredient, processing conditions, drying method, press tonnage, tablet weight, tablet thickness, hardness, friability and disintegration time. The “method” indicates whether the press was run at 18 rpm or at a slightly lower rpm resulting from hand operation of the press which increases the dwell time of the composition under pressure.

TABLE 1

| | | | | Processing | Pressure | | Press |
|---|---|---|---|---|---|---|---|
| Active | Active % | Excipient | Excipient % | Conditions | Process | Drying | (tons) |
| APAP | 75.0% | Del-Tab | 25.0% | Granulation | Wet | Oven | 0.75 |
| APAP | 81.4% | Del-Tab | 18.6% | Pressure Pulse 2× | | Oven | 0.5 |
| APAP | 81.4% | Del-Tab | 18.6% | Pressure Pulse 2× | | Oven | 0.75 |
| APAP | 85.0% | Del-Tab | 15.0% | Pressure Pulse 2× | | SD | 0.75 |
| APAP | 90.0% | Del-Tab | 10.0% | Pressure Pulse 2× | | Oven | 0.5 |
| APAP | 90.0% | Del-Tab | 10.0% | Pressure Pulse 2× | | Oven | 0.75 |
| APAP | 85.0% | National | 15.0% | Pressure Pulse 2× | | Oven | 1 |
| | | Starch 1551 | | | | | |
| APAP | 85.0% | Capsul | 15.0% | Pressure Pulse 2× | | Oven | 0.75 |
| APAP | 85.0% | Capsul | 15.0% | Pressure Pulse 2× | | Oven | 0.5 |
| APAP | 85.0% | National | 15.0% | Pressure Pulse 2× | | Oven | 0.5 |
| | | Starch 1500 | | | | | |
| APAP | 71.8% | National | 28.2% | Granulation | Wet | Oven | 1 |
| | | Starch 1500 | | | | | |
| Ibuprofen | 90.0% | Del-Tab | 10.0% | Pressure Pulse 2× | | Oven | 1 |
| Ibuprofen | 90.0% | Del-Tab | 10.0% | Untreated | | Oven | 1 |
| Ibuprofen | 90.0% | Del-Tab | 10.0% | Homogenize 10k | | Oven | 1 |
| | | | | RPM, 90 Sec | | | |
| Ibuprofen | 95.0% | Del-Tab | 5.0% | Pressure Pulse 2× | | Oven | 1 |
| Ibuprofen | 80.0% | Capsul | 20.0% | Pressure Pulse 2× | | Oven | 1 |

| | | Weight | Thickness | Hardness | Friablity | |
|---|---|---|---|---|---|---|
| Active | Method | (g/10 tablets) | (inches) | (kp) | (% loss) | DT (min) |
| APAP | | 0.805 | 0.225 | 10.97 | 0.38% | >30 |
| APAP | | 0.905 | 0.314 | 15.77 | 0.38% | >30 |
| APAP | | 0.990 | 0.298 | 11.79 | 0.40% | <37 |
| APAP | | 0.583 | 0.199 | 10.40 | 0.15% | 17 |
| APAP | Hand | 0.803 | 0.268 | 6.26 | 1.57% | 3.25 |
| APAP | Hand | 0.828 | 0.268 | 7.95 | 1.03% | 4.25 |
| APAP | Motor | 0.696 | 0.228 | 9.25 | 0.69% | 3 |
| APAP | Motor | 0.838 | 0.270 | 8.37 | 0.35% | >30 |
| APAP | Motor | 0.874 | 0.300 | 3.50 | 5.90% | 13 |
| APAP | Motor | 0.695 | 0.235 | 6.13 | 12.18% | 2.5 |
| APAP | | 0.850 | 0.258 | 15.40 | 0.14% | 8 |
| Ibuprofen | | 0.757 | 0.254 | 12.58 | 0.28% | >30 |
| Ibuprofen | | 0.797 | 0.260 | 10.35 | 0.18% | >30 |
| Ibuprofen | | 0.685 | 0.235 | 11.30 | 0.06% | ... |
| Ibuprofen | | 0.656 | 0.222 | 6.03 | 1.45% | 2 |
| Ibuprofen | | 0.626 | 0.216 | 6.67 | 0.69% | ... |

APAP = acetaminophen; SD = spray drying

### EXAMPLE 2

Table 2 presents the results of the practice of the present method using less than about 8 weight percent total excipient (relative to the total weight of the formulation) in the preparation of directly compressed acetaminophen tablets. The examples identified in Table 2 are prepared using an aqueous suspension containing acetaminophen (94 dry wt %), polysaccharide (Capsul® starch, 5 dry wt %) and non-ionic hydrophilic polymer (PVP, 1 dry wt %) mixed using a high speed homogenizer (Silverson homogenizer, Model No. L4RT at 9000 rpm). The homogenized mixture is then spray dried at 400° F. inlet and 200° F. outlet, using an atomizing spray nozzle resulting in a fine particle size. The dried homogenate is admixed with AcDiSol® NF (1 wt %) and magnesium stearate (0.5 wt %). The tablet formulation is compressed on the tableting press described in Example 1 at hand crank speed.

TABLE 2
>92% ACTIVE LOADED DIRECTLY COMPRESSED APAP TABLETS

| | | | | Hardness | Friability | |
|---|---|---|---|---|---|---|
| | | | | (kp) | (10 Tab | Disintegration |
| Pressure | APAP | Starch (%) | Polymer (%) | (average) | average) | (min) |
| 0.5 ton | 94.0% | Capsul 5.0% | PVP 1.0% | 13.63 | 2.475% | 2.18 |
| ″ | 94.5% | Capsul 5.0% | PVP 0.5% | 9.14 | 3.499% | 2.13 |
| ″ | 94.5% | Capsul 4.0% | PVP 1.5% | 12.92 | 1.468% | 9.03 |
| ″ | 95.0% | Capsul 4.0% | PVP 1.0% | 12.50 | 1.993% | 3.35 |
| ″ | 95.0% | Capsul 3.0% | PVP 2.0% | 9.35 | 2.004% | 4.30 |
| ″ | 96.5% | Capsul 3.0% | PVP 1.5% | 14.38 | 1.430% | 1.50 |
| ″ | 94.0% | Del-Tab 5.0% | PVP 1.0% | 7.71 | 4.031% | 3.00 |
| 1 ton | 94.0% | Capsul 5.0% | PVP 1.0% | 17.74 | 5.552% | 13.04 |
| ″ | 94.5% | Capsul 5.0% | PVP 0.5% | 8.14 | 2.967% | 1.29 |
| ″ | 94.5% | Capsul 4.0% | PVP 1.5% | 15.28 | 1.721% | 11.17 |
| ″ | 95.0% | Capsul 4.0% | PVP 1.0% | 8.71 | 20.183% | 10.05 |
| ″ | 95.0% | Capsul 3.0% | PVP 2.0% | 13.02 | 16.728% | 5.08 |
| ″ | 96.5% | Capsul 3.0% | PVP 1.5% | 9.68 | 19.274% | 6.40 |
| ″ | 94.0% | Del-Tab 5.0% | PVP 1.0% | 9.44 | 7.757% | 5.40 |

### EXAMPLE 3

### ULTRA HIGH PERCENTAGE ACTIVE DIRECTLY-COMPRESSED ACETAMINOPHEN TABLETS

The present method may be practiced by subjecting the tableting ingredients to pressure forces at different stages of handling. This example demonstrates the impact of such modification on the properties of the directly compressed acetaminophen tablets.

Pretreatment of Excipient-3A: The first embodiment passes the aqueous mixture (35 wt % solids) of starch and polymer through the pressure-pulse device into a holding/mixing tank, to which the difficult-to-compress active ingredient is added. The aqueous mixture of pressure pulse-treated starch and polymer is mixed with the active drug at room temperature with a paddle mixer to form a homogenous composition. This composition is kept at a constant room temperature, constantly mixed to keep the active ingredient from settling out to the bottom of the tank, and spray dried at 400° F. inlet and 200° outlet, using a co-current spray nozzle at a flow rate that forms a course product that is free-flowing and ready to punch on a tablet press at 18 rpm.

Complete Mixture Treatment -3B: The second embodiment passes the aqueous suspension (35 wt % solids) of active difficult-to-compress ingredient in admixture with the starch/polymer blend through the pressure pulse device. This pressure pulse-treated composition is kept at a constant room temperature, constantly mixed to keep the active ingredient from settling out to the bottom of the tank, and spray dried at 400° F. inlet and 200° outlet, using a co-current spray nozzle at a flow rate that forms a course product that is free-flowing and ready to punch on a tablet press at 18 rpm.

In each of embodiments 3A and 3B, the weight percent of materials used are as follows:

| INGREDIENTS | WT/BATCH | %/BATCH |
|---|---|---|
| Acetaminophen including 5 wt % Capsul ® | 295.5 | 98.5% |
| starch and 1 wt % PVP | | |
| Ac-Di-Sol | 3.0 | 1.0% |
| Magnesium Stearate | 1.5 | 0.5% |
| Total | 300.0 | 100% |

Table 3 presents the results of the practice of the present invention according to process embodiments 3A and 3B.

TABLE 3
>92% ACTIVE-LOADED DIRECTLY COMPRESSED APAP TABLETS -
PRESSURE-PULSE TREATED

| PROCESSING METHOD | Embodiment 3A | | | | Embodiment 3B | | | |
|---|---|---|---|---|---|---|---|---|
| Tablet Press Force (Tons) | 0.5 | 1 | 2 | 3 | 0.5 | 1 | 2 | 3 |
| Tablet weight (avg)(mg) | 885 | 882 | 881 | 868 | 882 | 875 | 870 | 870 |
| Tablet Hardness (avg) | 8.54 | 7.81 | 4.89 | 5.30 | 11.08 | 9.75 | 7.94 | 4.38 |
| Tablet Thickness (avg) | 0.28 | 0.27 | 0.27 | 0.26 | 0.28 | 0.27 | 0.27 | 0.27 |
| Friability | 45.50% | 0.53% | 0.41% | 1.60% | 0.58% | 0.49% | 1.00% | 1.38% |
| Start Weight | 8.76 | 7.53 | 7.37 | 8.74 | 8.74 | 8.75 | 8.68 | 8.70 |
| Final Weight | 4.78 | 7.49 | 7.34 | 8.60 | 8.69 | 8.71 | 8.59 | 8.58 |
| Disintegration Time (avg) | 3.15 | 6.14 | 16.09 | 19.20 | 5.07 | 12.45 | 16.10 | 16.55 |
| (minutes) | | | | | | | | |

Table 3 shows that the processing method influences the friability properties of the direct compression formulation prepared at compression forces of less than or about 2 tons, but are essentially the same above 2 tons. At 0.5 ton pressure force, embodiment 3A results in an etch unacceptable tablet friability, while the tablet prepared according to process embodiment 3B pressed at 0.5 ton exhibits an acceptable friability, hardness and disintegration time. At one ton pressure, both process embodiments produce directly compressed tablets with acceptable friability, hardness and disintegration times. As the compression force increases above two tons, the friability appears to increase and hardness decreases for both methods.

### EXAMPLE 4

When the processing lubricant is changed from magnesium stearate to stearic acid, the compressed tablets exhibit the properties presented in Table 4 below.

TABLE 4
>92% ACTIVE-LOADED DIRECTLY COMPRESSED APAP TABLETS -
PRESSURE - PULSE TREATED - STEARIC ACID

| PROCESSING METHOD | Process Embodiment 3A | | | | Process Embodiment 3B | | | |
|---|---|---|---|---|---|---|---|---|
| Tablet Press Force (Tons) | 0.5 | 1 | 2 | 3 | 0.5 | 1 | 2 | 3 |
| Tablet weight (avg)(mg) | 779 | 765 | 753 | 758 | 796 | 772 | 776 | 768 |
| Tablet Hardness (avg) | 8.84 | 9.00 | 4.27 | 3.96 | 11.74 | 10.84 | 9.37 | 6.68 |
| Tablet Thickness (avg) | 0.26 | 0.25 | 0.24 | 0.24 | 0.25 | 0.24 | 0.24 | 0.24 |
| Friability | 0.74% | 0.67% | 1.73% | 2.52% | 0.24% | 0.26% | 0.54% | 0.34% |
| Start Weight | 7.66 | 7.65 | 7.57 | 7.58 | 7.79 | 7.72 | 7.64 | 7.72 |
| Final Weight | 7.60 | 7.60 | 7.44 | 7.39 | 7.77 | 7.70 | 7.60 | 7.70 |
| Disintegration Time (avg) | 1.38 | 2.56 | 15.30 | 18.24 | 5.19 | 12.41 | 16.13 | 19.58 |
| (minutes) | | | | | | | | |

The change in tablet press lubricant significantly improves the friability of the process embodiment 3A pressed at 0.5 tons, but also resulted in the best overall hardness and friability of tablets prepared according to embodiment 3B tableted at 0.5 ton and 1 ton of pressure.

### COMPARATIVE EXAMPLE 5

A variety of polymers were incorporated into the acetaminophen formulations and found to be lacking in their ability to improve the compressibility of acetaminophen at the low levels achieved by the present invention. The results of these attempts are presented in Table 5 below. Each polymer additive is substituted for PVP in formulation prepared in accordance with process embodiment 3B.

APAP is mixed with either Capsule® starch (5 wt %) in combination with 1 wt % of the substitute polymer or with Capsul® starch (3 wt %) in combination with 1.5 wt % of the substitute polymer. The mixture is then processed in accordance with process embodiment 3B, mixed with AcDiSol® NF (1 wt %) and magnesium stearate (0.5 wt %) and then directly pressed in the rotary press at 18 rpm.

The use of 1 wt % or 1.5 wt % of cellulose-based polymers, hydrophobic polymers such as polyacrylates and surface active-type polymers (Tween) does not appear to assist in improving the compressibility of the difficult-to-compress ingredients.

TABLE 5
COMPARATIVE POLYMER EXCIPIENTS

| POLYMER | CMC 1% | | | EC 1% | | | EC 1.5% | | | HPC 1% | | | HPC 1.5% | | |
|---|---|---|---|---|---|---|---|---|---|---|---|---|---|---|---|
| Force (Tons) | 1 | 2 | 3 | 1 | 2 | 3 | 1 | 2 | 3 | 1 | 2 | 3 | 1 | 2 | 3 |
| Tablet weight | 779 | 765 | 753 | 796 | 772 | 766 | 734 | 730 | 701 | 644 | 603 | 657 | 721 | 692 | 707 |
| (mg) | | | | | | | | | | | | | | | |
| Hardness | 8.84 | 9.00 | 4.27 | 11.74 | 10.84 | 9.37 | 1.40 | 1.00 | 1.10 | 2.20 | 1.90 | 2.80 | 1.10 | 1.10 | 1.00 |
| (avg)(kp) | | | | | | | | | | | | | | | |
| Thickness | 0.26 | 0.25 | 0.24 | 0.25 | 0.24 | 0.24 | 0.24 | 0.24 | 0.23 | 0.22 | 0.21 | 0.22 | 0.24 | 0.27 | 0.24 |
| (avg) | | | | | | | | | | | | | | | |
| Friability (%) | 36.28 | 25.90 | 39.18 | 23.92 | 39.37 | 40.20 | 74.43 | 86.31 | 85.99 | 84.25 | 83.36 | 84.04 | 85.96 | 85.81 | 85.90 |
| Start Weight | 7.66 | 7.65 | 7.57 | 7.79 | 7.72 | 7.64 | 7.34 | 7.30 | 7.14 | 6.35 | 6.01 | 6.27 | 7.12 | 7.05 | 7.09 |
| Final Weight | 4.88 | 5.67 | 4.60 | 5.93 | 4.68 | 4.57 | 1.88 | 1.00 | 1.00 | 1.00 | 1.00 | 1.00 | 1.00 | 1.00 | 1.00 |
| Disintegration | 1.38 | 2.56 | 15.30 | 5.19 | 12.41 | 16.13 | 0.17 | 0.11 | 0.10 | 0.40 | 0.49 | 1.12 | 0.09 | 0.11 | 0.11 |
| Time | | | | | | | | | | | | | | | |
| (min)(avg) | | | | | | | | | | | | | | | |
| POLYMER | HPMC 1% | | | HPMC 1.5% | | | Eudragit 1.5% | | | Tween 60 1% | | | Eudragit 1% | | |
| Force (Tons) | 1 | 2 | 3 | 1 | 2 | 3 | 1 | 2 | 3 | 1 | 2 | 3 | 1 | 2 | 3 |
| Tablet weight | 671 | 674 | 699 | 646 | 722 | 718 | 722 | 718 | 735 | 741 | 765 | 776 | 738 | 767 | 785 |
| (mg) | | | | | | | | | | | | | | | |
| Hardness | 1.2 | 1.3 | 1.2 | 1.1 | 2.5 | 2.9 | 2.5 | 2.9 | 3.0 | 2.0 | 2.0 | 2.0 | 1.9 | 3.2 | 4.6 |
| (avg)(kp) | | | | | | | | | | | | | | | |
| Thickness | 0.23 | 0.22 | 0.24 | 0.22 | 0.24 | 0.24 | 0.24 | 0.24 | 0.24 | 0.24 | 0.24 | 0.25 | 0.24 | 0.26 | 0.25 |
| Friability (%) | 85.12 | 85.17 | 85.48 | 84.43 | 51.82 | 49.05 | 51.82 | 49.05 | 58.73 | 50.30 | 55.66 | 53.46 | 86.67 | | |
| Start Weight | 6.72 | 6.74 | 6.89 | 6.42 | 7.33 | 7.37 | 7.33 | 7.37 | 7.25 | 7.44 | 7.59 | 7.67 | 7.50 | 0.00 | 0.00 |
| Final Weight | 1.00 | 1.00 | 1.00 | 1.00 | 3.53 | 3.75 | 3.53 | 3.753 | 2.99 | 3.70 | 3.37 | 3.57 | 1.00 | 0.00 | 0.00 |
| Disintegration | 0.40 | 0.52 | 0.56 | 1.04 | 0.29 | 0.34 | 0.29 | 0.34 | 0.27 | 2.41 | 2.45 | 2.50 | 3.50 | 2.17 | 3.33 |
| Time | | | | | | | | | | | | | | | |
| (minutes) | | | | | | | | | | | | | | | |

“CMC” is sodium carboxymethylcellulose, “EC” is ethyl cellulose, “HPC” is hydroxypropyl-cellulose, “HPMC” is hydroxymethylpropylcellulose, Tween ® 60 is polyoxyethylene sorbitan fatty acid ester, and “Eudragit ® ” is a water insoluble cationic copolymer of dimethylaminoethyl methacrylate and methacrylic esters.

We claim:

1. A method for direct compression of particles comprising the steps of
(A) subjecting a suspension comprising particles of an active material and a compression excipient in a fluid medium to a pressure force to form a suspension of modified particles;
(B) removing said modified particles from said fluid medium to form a dried homogenate; and
(C) directly compressing said dried homogenate.

2. A method according to claim 1 wherein said active material exhibits poor binding ability.

3. A method according to claim 1 wherein said compression excipient is a polysaccharide.

4. A method according to claim 3 wherein said polysaccharide is a modified starch.

5. A method according to claim 1 wherein said liquid is water.

6. A method according to claim 1 wherein said pressure-force means imparts a shear, abrupt pressure and/or cavitation forces sufficient to intimately admix said active material and said excipient.

7. A method according to claim 6 wherein said fluid medium is removed by the application of heat to form said dried homogenate.

8. A method according to claim 7 wherein said fluid medium is removed by spray drying to form said dried homogenate.

9. A method according to claim 1 wherein said dried homogenate is directly compressed to form a USP acceptable tablet with a hardness from about 6 to about 30 kp.

10. A method according to claim 1 wherein said excipient comprises from 100 to about 85% polysaccharide and 0 to about 15% of a hydrophilic polymer.

11. A method according to claim 10 wherein said active ingredient comprises from about 82 to about 99 percent active ingredient.

12. A method according to claim 1 wherein a lubricant is added to said dried homogenate before direct compression.

13. A method according to claim 12 wherein said lubricant is a pharmaceutically acceptable fatty acid or a pharmaceutically acceptable fatty acid salt or ester.

14. A method according to claim 6 wherein said pressure-force comprises the application of a pressure-pulse forcing said suspension through a chamber that imparts cavitation and shear forces to said suspension.

15. A method for direct compression of difficult-to-compress materials comprising the steps of
(A) subjecting a suspension comprising difficult-to-compress active ingredient material particles and excipient comprising polysaccharide and a non-ionic hydrophilic polymer in a fluid medium to a pressure force to form a homogenate US suspension;
(B) removing said fluid medium from said homogenate; and
(C) directly compressing said dried homogenate to form a USP acceptable tablet.

16. A method according to claim 15 wherein said fluid medium is removed by spray is drying.

17. A method according to claim 16 wherein said fluid medium is water and said dried homogenate comprises from about 1 to about 8 per cent remaining moisture.

18. A method according to claim 17 wherein said USP acceptable tablet exhibits a hardness between about 6 kp and about 30 kp, and friability of less than about 3%.

19. A method for the direct compression of difficult-to-compress materials comprising
(A) preparing an aqueous suspension comprising particles of a difficult-to-compress active ingredient and an water soluble excipient composition;
(B) subjecting said suspension to an abrupt pressure change to form a pressure-treated suspension;
(C) removing substantially all of the water in said pressure-treated suspension to form a dried suspendant; and
(D) directly compressing said dried suspendant.

20. A method according to claim 19 wherein said abrupt pressure change imparts shear and/or cavitation to said suspended particles sufficient to form an ultra-homogenized suspension.

21. A method according to claim 20 wherein said ultra-homogenized suspension is dried using heat, vacuum, or heat and vacuum, or in a spray dryer or in a fluid bed.

22. A method according to claim 21 wherein said excipient composition includes a polysaccharide.

23. A method according to claim 22 wherein said polysaccharide is a starch or modified starch.

24. A method according to claim 23 wherein said difficult-to-compress material exhibits compression properties of crystalline powders.

25. A method according to claim 24 wherein said excipient comprises from about 4 to about 18 percent by weight of the tablet.

26. A method according to claim 25 wherein said excipient composition comprises a water soluble polysaccharide and a non-ionic hydrophilic polymer.

27. A method according to claim 26 wherein said excipient composition comprises from 99.9 to about 85% polysaccharide and 0.1 to about 15% of a nonionic lactam-containing hydrophilic polymer; and wherein said active ingredient comprises from about 82 to about 99 weight percent of said tablet.

28. A method for reducing the amount of excipient required to prepare a compressed form including a difficult-to-compress active ingredient, comprising
(A) preparing a suspension of said difficult-to-compress active ingredient in a first amount and a polysaccharide excipient in a second amount in an aqueous
(B) wherein said second amount is at least about 30% less than a third amount that is required by a wet granulation process to form a compressible form with said active ingredient; and
(C) subjecting said suspension to sufficient shear and/or cavitation forces such that the resulting suspendant particles, after drying, are capable of direct compression into USP acceptable tablets;
(D) recovering said resulting particles from said suspension aqueous medium;
(E) compressing said recovered particles to form a USP acceptable tablet.

29. A method according to claim 28 wherein said tablet is made in a press with a force of from about 0.5 to about 3.0 tons of pressure.

30. A method according to claim 29 wherein said USP tablet is prepared without the occurrence of capping or laminating.

31. A method according to claim 30 wherein said acceptable compression form exhibits a hardness from about 6 kp to about 30 kp.

32. A method according to claim 31 wherein said tablet disintegrates in aqueous medium in less than about 45 minutes.

33. In a direct tablet compression method comprising preparing a wet mixture of active ingredient and excipient in water, drying said mixture and optionally admixing said mixture with a lubricant and subjecting the resultant mixture to a pressure of between about 0.5 to about 3 tons, the improvement comprising performing the steps of the method according to claim 28.

34. A method for direct compression of difficult-to-compress active ingredients comprising
(A) preparing an aqueous suspension of particles comprising said active ingredient and polysaccharide excipient wherein said excipient comprises from about 4 to about 18 percent of the total dry weight of said particles; and
(B) subjecting said suspension to sufficient shear and/or cavitation forces to form a particle admixture capable of direct compression into USP acceptable tablets.

35. A method according to claim 34 wherein said tablet is made in a press with a force of from about 0.5 to about 3.0 tons of pressure.

36. A method according to claim 35 wherein said acceptable compression form is prepared without the occurrence of capping or laminating.

37. A method according to claim 36 wherein said acceptable compression form exhibits a hardness from about 6 kp to about 30 kp.

38. A method according to claim 37 wherein said tablet disintegrates in aqueous medium in less than 45 minutes.

39. A solid dosage form comprising from about 82 to about 99 percent of an active ingredient that is difficult-to-compress and about 1 to about 18 per cent of a polysaccharide excipient and exhibiting a hardness of from about 6 kp to about 30 kp, an acceptable friability and an aqueous disintegration time less than about 45 minutes.

40. A solid dosage form according to claim 39 wherein said active ingredient is a material that exhibits compression properties of the type exhibited by crystalline powder materials such as acetaminophen and ibuprofen.

41. A solid dosage form prepared by the process of claim 1.

42. A process for the preparation of a direct-compressed tablet excipient comprising
(A) admixing an aqueous dispersion of polysaccharide and non-ionic lactam containing-hydrophilic polymer, and
(B) subjecting said mixture to a pressure-pulse force.

43. A compression excipient prepared according to claim 42.

44. A process for the direct compression of a solid tablet form comprising admixing the compression excipient according to claim 42 with an aqueous suspension or solution of difficult-to-compress active ingredient, homogenizing said admixture and spray drying the combined mixture.

45. The process according to claim 44 wherein said polymer is polyvinylpyrrolidone.
